(19) United States
(12) Patent Application Publication
(10) Pub. No.: US 2010/0291173 A1
(43) Pub. Date: Nov. 18, 2010

(54) METHOD OF IMPROVING RENAL FUNCTION

(75) Inventors: Myron Spector, Brookline, MA (US); Peter Geistlich, Stansstad (CH)

Correspondence Address: ROTHWELL, FIGG, ERNST & MANBECK, P.C., 1425 K STREET, N.W., SUITE 800, WASHINGTON, DC, 20005, US

(73) Assignee: ED. GEISTLICH SOEHNE AG FUER CHEMISCHE INDUSTRIE, Wolhusen (CH)

(21) Appl. No.: 12/438,253

(22) PCT Filed: Sep. 4, 2007
(86) PCT No.: PCT/US07/19262

§ 371 Date: Jul. 26, 2010

Related U.S. Application Data
(60) Provisional application No. 60/842,155, filed on Sep. 5, 2006.

(51) Int. Cl. A61K 31/7088; A61K 9/50; A61K 9/00; A61F 2/00; A61P 13/12

(52) U.S. Cl. 424/423; 514/44 R; 424/501; 424/484; 424/499

(57) ABSTRACT

A method of improving renal function in a mammal suffering from, or at risk of developing, at least partial renal failure or renal dysfunction, includes administering to renal tissue of the mammal, a combination comprising a non-viral vector comprising a non-viral particulate carrier which carries a therapeutically effective amount of genetic material capable of expressing a renal function-enhancing Osteogenic Protein-1/Bone Morphogenic Protein-7 (OP-1/BMP-7) polypeptide in the renal tissue.

### CROSS-REFERENCE TO RELATED APPLICATIONS

This application is a 35 U.S.C. §371 National Phase Entry Application from PCT/US2007/019262, filed Sep. 4, 2007, and designating the United States. This application also claims the benefit of U.S. patent application Ser. No. 60/842,155, filed Sep. 5, 2006; the disclosures of which are incorporated herein in their entirety by reference.

### FIELD OF THE INVENTION

The present invention relates to the field of at least partial prevention and/or treatment of acute and/or chronic renal failure and/or renal dysfunction in mammals.

### BACKGROUND OF THE INVENTION

The mammalian renal system serves primary roles both in the removal of catabolic waste products from the bloodstream and in the maintenance of fluid and electrolyte balances in the body. Renal failure is, therefore, a life-threatening condition in which the build-up of catabolites and other toxins, and/or the development of significant imbalances in electrolytes or fluids, may lead to the failure of other major organs systems and death. As a general matter, renal failure is classified as “acute” or “chronic”. As detailed below, acute and chronic renal failure are debilitating and life-threatening diseases for which no adequate treatments exist to delay, and/or reverse kidney structural alterations associated with the disease.

Acute renal failure (ARF) is usually caused by an ischemic or toxic insult that results in an abrupt decline in renal functions. The kidneys are highly susceptible to ischemia and toxicants because of their unique anatomic and physiologic features. The large renal blood flow (approximately 25% of the cardiac output) results in increased delivery of blood-borne toxicants to the kidney as compared to other organs. The renal cortex is especially susceptible to toxicant exposure because it receives 90% of renal blood flow and has a large endothelial surface area due to the numerous glomerular capillaries. Within the renal cortex, the proximal tubule (the S3 segment or “pars recta”) and the epithelial cells of the thick ascending arm of the loop of Henle, are most frequently affected by ischemic and toxicant-induced injury because of their solute transport functions and high metabolic rates. As water and electrolytes are reabsorbed from the glomerular filtrate, tubular epithelial cells can be exposed to increasingly high concentrations of toxicants. Similarly, in the medulla the counter-current multiplier system may concentrate toxicants. Toxicants that are either secreted or reabsorbed by tubular epithelial cells (such as gentamicin) may accumulate in high concentrations within these cells. Finally, the kidneys also play a role in the biotransformation of many drugs and toxicants. Biotransformation usually results in the formation of metabolites that are less toxic than the parent compound; however, in some cases (such as oxidation of ethylene glycol to glycolate and oxalate) the metabolites are more toxic.

ARF has three distinct phases, which are categorized as initiation, maintenance, and recovery. During the initiation phase, therapeutic measures that reduce the renal insult (e.g., fluid therapy) can prevent the development of established ARF. The maintenance phase is characterized by tubular lesions and established nephron dysfunction. The recovery phase of ARF occurs when renal function improves subsequent to nephron repair and compensatory hypertrophy. Tubular lesions may be repaired if the tubular basement membrane is intact and viable cells are present. In addition, functional and morphologic hypertrophy of surviving nephrons can, in some cases, adequately compensate for decreased nephron numbers. Even if renal functional recovery is incomplete, adequate function may be re-established in some cases. More commonly, however, tubular damage is severe and irreversible and a large percentage of animals die or are euthanized in the maintenance phase of ARF.

Despite tremendous efforts to decipher the cellular and molecular pathogenesis of ARF during the past decades, no effective treatment is currently available and the incidence of mortality remains very high in veterinary medicine. At least two retrospective studies have documented the poor prognosis associated with ARF in dogs. In a study of hospital acquired ARF, the survival rate was 38%, whereas in another study of all types of ARF, the survival rate was 24%. Thus, there is an un-met medical need for improved prevention and/or treatment of ARF.

Chronic renal failure (CRF) may be defined as progressive, permanent and significant reduction of glomerular filtration rate (GFR) due to significant and continuing loss of nephrons. CRF typically begins from a point at which a chronic renal insufficiency (i.e., a permanent decrease in renal function of at least 50-60%) has resulted from some insult to the renal tissues, which has caused a significant loss of nephron functional units. The initial insult may not have been associated with an episode of acute renal failure. Irrespective of the nature of the initial insult, CRF manifests a “final common path” of signs and symptoms as nephrons are progressively lost and GFR progressively declines. This progressive deterioration in renal function is slow and seemingly inevitable, typically spanning several months to years in canine and feline subjects and many decades in human patients.

The early stage of CRF typically begins when GFR has been reduced to approximately one-third of the normal level (e.g., 30-40 ml/min for an average human adult). As a result of the significant nephron loss, and in an apparent “attempt” to maintain the overall GFR with fewer nephrons, the average single nephron GFR(SNGFR) is increased by adaptation of the remaining nephrons at both the structural and functional levels. One structural manifestation of this adaptation that is readily detectable by microscopic examination of biopsy samples is a “compensatory hypertrophy” of both the glomeruli and the tubules of the kidney, a process that actually increases the volume of filtrate which can be produced by each remaining nephron by literal enlargement of the glomeruli and tubules.

As a result of the hypertrophy or dilatation of the collecting ducts, the urine of subjects with CRF often contains casts which are 2-6 times the normal diameter (referred to herein as “broad casts” or “renal failure casts”. The presence of such broad casts aids in diagnosis of CRF. At the same time, there are functional changes in the remaining nephrons, such as decreased absorption or increased secretion of normally excreted solute, which may be responses to hormonal or paracrine changes elsewhere in the body (e.g., increasing levels of parathyroid hormone (PTH) in response to changes in serum levels of calcium and phosphate).

These adaptations in the early stage CRF are not successful in completely restoring GFR or other parameters of renal function and, in fact, subject the remaining nephrons to increased risk of loss. For example, the increased SNGFR is associated with mechanical stress on the glomerulus due to hypertension and hyperperfusion. he loss of integrity of podocyte junctures leads to increased permeability of the glomerulus to macromolecules or “leakiness” of the glomerular capsule. Proliferative effects are also observed in mesangial, epithelial and endothelial cells, as well as increases in the deposition of collagen and other matrix proteins. Sclerosis of both the glomeruli and tubules is another common symptom of the hypertrophied nephrons and the risk of coagulation in the glomerulus is increased. In particular, these adaptations of the remaining nephrons, by pushing the SNGFR well beyond its normal level, actually decrease the capacity of the remaining nephrons to respond to acute changes in water, solute, or acid loads, and therefore actually increase the probability of additional nephron loss.

As CRF progresses, and GFR continues to decline to less than 10% of normal (i.e., around 5-10 ml/min in humans), the subject enters into end-stage renal disease (ESRD). During this phase, the inability of the remaining nephrons to adequately remove waste products and maintain fluid and electrolyte balance, leads to a rapid decline in which many organ systems, and particularly the cardiovascular system, may begin to fail. At this point, renal failure will rapidly progress to death unless the patient receives renal replacement therapy (i.e., chronic hemodialysis, continuous peritoneal dialysis, or kidney transplantation).

The management of CRF must be conducted to ameliorate all identifiable clinical, metabolic, endocrine and biochemical consequences induced by renal failure including, but not limited to, azotemia, nutritional inadequacies, hypoproliferative anaemia, disordered mineral metabolism, electrolyte disturbances, metabolic acidosis, proteinuria, disordered water metabolism, systemic hypertension and the progression of renal injury through interstitial fibrosis that is considered to be the commonly converging outcome of CRF regardless of the specific etiology.

While tremendous progress has been made to address several clinical, metabolic, endocrine and biochemical consequences of CRF, the therapy of clinically chronic fibrosis remains extremely challenging and therefore the long-term medical control of renal disease remains an important un-met therapeutic need. Certain therapy targeting the reduction of renal disease-associated fibrosis is focused on the reduction of the activity of the renin-angiotensin system (RAS). Although this strategy has been shown to slow the disease evolution, its efficacy remains partial and it does not completely halt the progression of chronic fibrosis in experimental and clinical conditions. This is probably because many factors other than RAS contribute to the pathogenesis of CRF associated fibrosis.

The prevalence of CRF in cats and dogs is increasing. For every 1000 cats evaluated in 1980 in the US, four had renal failure regardless of age. By 1990, the number of reported cases of renal failure has quadrupled with 16 cases identified for every 1000 cats examined. For cats older than 15 years of age, 153 cases of renal failure were diagnosed in 1990 for every 1000 examinations. The increase in prevalence of renal failure in aging cats may reflect an increase in veterinary care sought by owners as well as greater efforts by veterinarians to detect the disease. Whatever the reason, these findings emphasize the emerging awareness and importance of CRF in older animals. The most frequent etiologies of CRF in companion animals include, but are not limited to, idiopathic chronic interstitial nephritis, irreversible ARF, familial renal dysplasia or aplasia, congenital polycystic kidney disease, amyloidosis, glomerulonephritis, hypercalcemia, bilateral hydronephrosis, leptospirosis, pyelonephritis, nephrolithiasis bilateral, Falconi-like syndrome, hypertension, renal lymphosarcoma.

In human medicine, approximately 600 patients per million receive chronic dialysis each year in the USA, at an average cost approaching $60,000-$80,000 per patient per year. Of the new cases of end-stage renal disease each year, approximately 28-33% are due to diabetic nephropathy (or diabetic glomerulopathy or diabetic renal hypertrophy), 24-29% are due to hypertensive nephrosclerosis (or hypertensive glomerulosclerosis), and 15-22% are due to glomerulonephritis. The 5-year survival rate for all chronic human dialysis patients is approximately 40%, but for patients over 65, the rate drops to approximately 20%. Therefore, a need remains for treatments to prevent the progressive loss of renal function which has caused almost 200,000 human patients in the USA alone to become dependent upon chronic dialysis, and which results in the premature deaths of tens of thousands each year.

In light of the fact that specific morphogens and/or growth factors that exhibit renotropic properties and promote tubular repair and recovery of renal function have been recently identified, it is conceivable that some of these molecules may be used as therapeutic agents for the prevention and/or treatment of ARF and/or CRF. One such agent is Bone Morphogenetic Protein-7 (BMP-7, or Osteogenic Protein-1, OP-1), which is a member of the Transforming Growth Factor-.beta.(TGF-.beta.) superfamily. BMP-7 binds to activin receptors types I and II, but not to TGF-.beta. receptors type I, II and III. Monomeric BMP-7 has a molecular weight of 17 to 19 kDa and was originally identified by its ability to induce ectopic bone formation. BMP-7 polypeptide is secreted as a homodimer with an apparent molecular weight of approximately 35-36 kDa. Recently, BMP-7 has been shown to be a key morphogen during nephrogenesis. Renal expression of BMP-7 continues in mature kidneys, especially in medullary collecting ducts. Renal tubules also express BMP-7 receptors. In animal models of ARF and CRF, renal expression of BMP-7 is significantly down-regulated and the administration of recombinant BMP-7 protein has been reported to accelerate renal recovery, an effect that was associated with less interstitial inflammation and programmed cell death.

However, because BMP-7 has a short half live in vivo (approximately 30 min), maintenance of a sustained level of exogenous protein in the circulation following injection of the purified protein requires multiple short-interval administrations, creating a very significant practical challenge. The cost of such a multi-injection therapy is too high to be applicable in veterinary medicine.

There remains a need in the art for methods of treating renal dysfunction.

### SUMMARY OF THE INVENTION

A method of improving renal function in a mammal suffering from, or at risk of developing, at least partial renal failure or renal dysfunction, comprises administering to renal tissue of said mammal, a combination comprising a non-viral vector comprising a non-viral particulate carrier which carries a therapeutically effective amount of genetic material capable of expressing a renal function-enhancing Osteogenic Protein-1/Bone Morphogenic Protein-7 (OP-1/BMP-7) polypeptide in said renal tissue.

### DETAILED DESCRIPTION OF THE INVENTION

The present invention is directed to methods of prevention and treatment of mammalian subjects who are suffering from, or who are at risk of, acute or chronic renal failure, and to non-viral vectors and pharmaceutical compositions for use in such methods. The methods, vectors and compositions of the invention are useful for reducing mortality and/or morbidity rates, and preventing, inhibiting, delaying, or alleviating the progressive loss of renal function which characterizes renal failure. Subjects for which the methods, non-viral vectors, and compositions of the present invention are useful include, but are not limited to, subjects already afflicted with acute or chronic renal failure, subjects who have already received renal replacement therapy, as well as any subject reasonably expected to suffer from an acute or progressive loss of renal function associated with progressive loss of functioning nephron units. Whether a particular subject is at risk of renal disease, and/or whether a subject may benefit from the methods and/or compositions of the present invention, is a determination that can be routinely made by one of ordinary skill in the relevant medical or veterinary art.

OP-1, also known as BMP-7, is a member of a class of naturally occurring growth factors called bone morphogenetic proteins (BMPs).

In accordance with one embodiment, a method of improving renal function in a mammal suffering from, or at risk of developing, at least partial renal failure or renal dysfunction, comprises administering to renal tissue of the mammal a combination comprising a non-viral vector comprising a non-viral particulate carrier which carries a therapeutically effective amount of a genetic material capable of expressing a renal function-enhancing OP-1/BMP-7 polypeptide in the renal tissue (e.g., renal cells). In preferred embodiments, the non-viral vector comprises polymer particles. Preferably, the polymer is cationic, most preferably is a natural polymer and most preferably, the polymer comprises chitosan.

In preferred embodiments, the particles have a size within a range of about 5-3,000 nm, more preferably within a range of about 10-1000 nm, and still more preferably within a range of about 15-700 nm. In some embodiments, the particles have a substantially uniform size of less than about 500 nm.

The OP-1/BMP-7 polypeptide genetic material can be incorporated into the carrier material (e.g., polymeric carrier material) by mixing. When chitosan is utilized as the polymeric material, the weight ratios of chitosan to the OP-1/BMP-7 genetic material may be, for example, 0.5:1, 1:1, 2:1, 5:1, 10:1, 20:1, 40:1, or any suitable ratio.

In certain embodiments, the particles comprise nanoparticles.

In preferred embodiments, the genetic material comprises a DNA plasmid, capable of in vivo expression of OP-1/BMP-7 polypeptide.

In particularly preferred embodiments, the inventive particles carrying OP-1/BMP-7 genetic material is administered directly to a kidney of a mammal. In certain embodiments, the inventive particles are in a composition wherein the particles are suspended in a pharmaceutically or veterinarily acceptable carrier. Such a composition can be injected directly into renal tissue or a kidney of a mammal, or otherwise are administered to the mammal by injection or infusion.

Alternatively, the inventive particles can be present in a matrix which is contacted with renal tissue. The matrix can be implanted within a kidney or renal tissue of a mammal, or the matrix can be attached to a surface of a kidney, e.g., by sutures or adhesive such as fibrin glue.

When a matrix is used, the matrix preferably comprises collagen, e.g., a collagen sponge. The collagen of the matrix may comprise any suitable collagen, e.g., collagen I, collagen II, collagen III or a combination thereof.

In certain embodiments, the matrix further comprises stem cells capable of differentiating into renal cells.

In certain embodiments, the matrix may include taurolidine, taurultam, a mixture thereof, or an equilibrium thereof, in a weight percentage as compared to the matrix of, e.g., 1-5%, more preferably about 2-4%. The taurolidine and/or taurultam facilitates reduction in TGF_{beta}, and assists in bringing about a healthy balance between BMP-7 and TGF_{beta} in renal tissue.

In certain embodiments, substantially uniform particles of less than 250 nm of spherical shape are preferred.

OP-1/BMP-7 plasmids are known in the art and available for use in the present invention, or easily adaptable for such use by well known techniques. OP-1/BMP-7 plasmids are disclosed, for example, in Fang et al., Proc. Nat. Acad. Sci. USA, 93:5753-5758 (June 1996), and Bright et al., Spine, 31(10):2163-2172, September 1, 2006. Suitable plasmids also can be produced using well known techniques. DNA sequences encoding OP-1/BMP-7 polypeptides are disclosed in U.S. Pat. No. 5,141,905. OP-1/ BMP-7 polypeptides are disclosed in, for example, U.S. Pat. Nos. 5,366,875, 6,861,404 and 7,196,056, as well as U.S. Patent Application Publication No. 2005/0143304 A1.

In an embodiment where the non-viral vector particles are present in a matrix which is contacted with renal tissue, the matrix may include stem cells present therein, e.g., renal stem cells, capable of differentiating into renal cells. It is known that metanephric mesenchyme contains embryonic renal stem cells, Oliver et al., Am J Physiol Renal Physiol 283:F799-F809, 2002.

In one embodiment the present invention relates to a non-viral vector containing and expressing in a host a pre-pro BMP-7 gene, a proBMP-7 gene or a mature BMP-7 gene. The BMP-7 gene encoding the pre-proBMP-7 polypeptide, the proBMP-7 polypeptide or the mature BMP-7 polypeptide may originate from a mammal. In a preferred embodiment, the expression vector may comprise a polynucleotide that encodes a pre-proBMP-7, a pro-BMP-7 or a mature BMP-7 polypeptide. The polynucleotide encoding the BMP-7 polypeptide may be operatively linked to a promoter and optionally an enhancer.

In one embodiment, the invention relates to a non-viral vector containing and expressing the proBMP-7 polypeptide, wherein the proBMP-7 polypeptide is deleted of the “pre” peptide at the N-terminus, and wherein a peptide signal sequence from a different origin is fused to the proBMP-7 polypeptide. Advantageously, the peptide signal sequence may be the insulin-like growth factor 1 (IGF-1) or the tissue plasminogen activator (tPA) peptide signal sequence. In another embodiment, the expression vector may comprise a polynucleotide that encodes a mature BMP-7 polypeptide wherein said polypeptide is fused with a peptide signal sequence from BMP-7, IGF-1 or tPA.

In another embodiment the invention relates a non-viral vector expressing a pre-proBMP-7 polypeptide, a proBMP-7 polypeptide or a mature BMP-7 polypeptide and a pharmaceutically or veterinarily acceptable carrier, excipient or vehicle. In a particular embodiment, the pharmaceutical composition may comprise a substance to improve the efficacy of transmission of the vector into the host cells.

In yet another embodiment the invention relates to a method for delivering the BMP-7 polypeptide to a mammal which may comprise injecting a vector capable of expressing, in vivo, a pre-proBMP-7 polypeptide, a proBMP-7 polypeptide or a mature BMP-7 polypeptide. In an advantageous embodiment, the animal host may be a human, a dog or a cat. The invention relates to the use of such a vector to prevent and/or treat a mammal for chronic or acute renal failure. The pharmaceutical compositions of the invention may be administered by any suitable route of administration including, but not limited to, directly to renal tissue, or by the intramuscular or subcutaneous route.

In a further embodiment the invention relates to the use of pharmaceutical compositions according to the present invention to treat mammals exhibiting an increase of in serum creatinine concentration and/or an increase in serum urea nitrogen concentration.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

The methods and compositions of the present invention can be used for at least partly preventative treatment of renal failure. The terms “prevention”, “prophylaxis”, “preventative treatment” and “prophylactic treatment”, as they relate to renal failure, and as they are used herein and in the field of human and veterinary medicine, relate to the treatment of either healthy subjects suffering from an unrelated disease, but who are considered to be at risk of acute renal failure. Risk factors for acute renal failure in mammals include, but are not limited to, shock and/or hypovolemia (for example haemorrhage, hypotensive shock, septic shock, prolonged or deep anaesthesia, hypovolemia, heat stroke, trauma, burns, or diuretic abuse), systemic diseases (for example pancreatis, peritonitis, hepatic failure, disseminated intravascular coagulation, adrenal insufficiency or vasculitis), ischemia (as caused by, for example, thromboembolic occlusion or malignant hypertension), infections (for example leptospirosis, pyelonephritis, infectious peritonitis, borreliosis, leishmaniasis, babesiosis, septicaemia or septic emboli), systemic renal disease (for example multiple organ failure, glomerulonephritis, systemic lupus erythematosus, renal vein thrombosis, urinary outflow obstruction, haemolytic uremic syndrome, hemepigmenturia-crush syndrome or polycythemia), advanced age, congenital and/or genetic renal diseases, and other miscellaneous factors such as exposure to nephrotoxins (for example aminoglycosides, amphotericin B, cisplatin, adriamycin, non steroidal anti-inflammatory drugs, diuretics, IL-2 or allopurinol), neoplasia (for example lymphoma), hypercalcemia, trauma (for example avulsions), malignant hypertension, oxalate nephrosis, and the like.

Treatment for at least partly preventative purposes is generally conducted within a few weeks (ideally with 6 to 8 days) before the exposure of a healthy subject to one or more of the aforementioned risk factors for acute renal failure. Alternatively, in diseased subjects for which an associated risk factor for acute renal failure has been identified, treatment may be conducted as quickly as possible to limit any negative impact of the primary disease of risk factor on the kidney metabolism and/or the structure and organization of the kidney tissue.

In addition to preventative treatments, the methods and compositions of the present invention can also be used for therapeutic treatment of renal failure. The terms “therapy” or “therapeutic treatment”, as they relate to renal failure, and as they are used herein and in the field of human or veterinary medicine, relate to treating, or supporting and/or accelerating treatment of, subjects that are already suffering from, or are recovering from (i.e. are in the recovery phase) acute renal failure, or treatments aimed at slowing down and/or reversing lesion evolution in subjects diagnosed as having, or at being at risk of, chronic renal failure. A critical objective of therapy is to reduce the risk of an evolution towards CRF subsequent to an ARF event. As used herein, a subject is said to suffer from CRF, or be at risk of developing CRF, if the subject is reasonably expected to suffer a progressive loss of renal function associated with progressive loss of functioning nephron units. Whether a particular subject suffers of CRF, or is at risk of developing CRF, can readily be determination by one with ordinary skill in the relevant veterinary or medical art.

Risk factors for chronic renal failure may include, but are not limited to, idiopathic chronic interstitial nephritis, irreversible ARF, familial renal dysplasia or aplasia congenital kidney disease amyloidosis, glomerulonephritis, hypercalcemia, bilateral hydronephrosis, leptospirosis, pyelonephritis, nephrolithiasis bilateral, Falconi-like syndrome, and hypertension.

Risk factors for chronic renal failure may include, but are not limited to, idiopathic chronic interstitial nephritis, irreversible ARF, renal lymphosarcoma, kidney disease, glomerulonephritis, bilateral hydronephrosis, amyloidosis, pyelonephritis, hypercalcemia, and bilateral nephrolithiasis.

Human subjects suffering from CRF, or whom are at risk of developing CRF, or who may be in need of renal replacement therapy, include, but are not limited to, subjects with end-stage renal disease, chronic diabetes nephropathy, hypertensive nephrosclerosis, chronic glomerulonephritis, hereditary nephritis, and/or renal dysplasia, subjects who have had a biopsy indicating glomerular hypertrophy, tubular hypertrophy, chronic glomerulosclerosis, and/or chronic tubulo-interstitial sclerosis, subjects who have had an ultrasound, MRI, CAT scan, or other non-invasive examination indicating the presence of renal fibrosis, subjects having an unusual number of broad casts present in their urinary sediment, subjects having a glomerular filtration rate (“GFR”) which is chronically less than 50%, and more particularly less than about 40%, 30% or 20%, of the expected GFR for the subject, subjects possessing a number of functional nephron units which is less than about 50%, and more particularly less than about 40%, 30% or 20% of the number of functional nephron units possessed by a healthy but otherwise similar subject, subjects with only a single kidney, and subjects that are kidney transplant recipients.

The “glomerular filtration rate” or “GFR” is proportional to the rate of clearance into the urine of “marker” substance which is a plasma-borne substance which is not bound by serum proteins, is freely filtered across glomeruli, and is neither secreted nor reabsorbed by the renal tubules. Optionally, the GFR can BE corrected for body surface area. The preferred marker substance for GFR measurements is inulin, however, because of difficulties in measuring the concentration of this substance, creatinine is typically used as the marker for GFR measurements in clinical settings.

An estimate of the “expected GFR” may be provided based upon considerations of a subject's age, weight, sex, body surface area, and degree of musculature, and the plasma concentration of some marker compound (e.g., creatinine) as determined by a blood test. Because creatinine is produced by striated muscles, the expected GFR of human females subjects is estimated by the same equation multiplied by 0.85 to account for expected difference in muscle mass.

Microscopic examination of urinary sediment for the presence of formed elements is a standard procedure in urine analysis. Amongst the formed elements which may be present in urine, are cylindrical masses of agglutinated materials that typically represent a mold or “cast” of the lumen of a distal convoluted tubule or collecting tube. In healthy human beings, such casts typically have a diameter of 15-25 um. In subjects with CRF, however, hypertrophy of the tubules may result in the presence of casts which are 2-6 times the diameter of normal casts and often have a homogeneous waxy appearance. These are referred to as “broad casts” or “renal failure casts”. As used herein, the term “broad cast” is used to refer to urinary sediment casts having a diameter of 2-6 times normal for the subject, or about 30-150 um for human casts.

As used herein with respect to clinical indications the term “acute” is used to refer to renal pathologies for which onset occurs rapidly, typically within hours or days of exposure to an insult or risk factor.

As used herein with respect to clinical indications the term “chronic” means persisting for a period of at least three, and more preferably, at least six months. Thus, for example, a subject with a measured GFR chronically below 50% of GFR.sub.exp is a subject in which the GFR has been measured and found to be below 50% of GFR.sub.exp in at least two measurements separated by at least three, and more preferably, by at least six months, and for which there is no medically sound reason to believe that GFR was substantially (e.g., 10%) higher during the intervening period. Other indicators of abnormal renal function, such as the presence of broad casts, could similarly be described as chronic if the presence of such indicators persisted in at least two measurements separated by at least three, and more preferably, by at least six months.

The present invention provides therapies and preventative treatments for renal failure that utilize pharmaceutical compositions comprising genetic material capable of expressing an OP-1/BMP-7 polypeptide in vivo and methods for inducing a sustained increase in an OP-1/BMP-7 polypeptide concentration and thereby reducing the activation of the TGF-.beta. pathway in cells. TGF-.beta. activation triggers, amongst other things, the phospohorylation of Smad2 and Smad3 factors and their nuclear import, leading to the promotion of epithelial-mesenchymal transition and to the repression of mesenchymal-epithelial transition, and acting as key trigger for fibrosis. Although BMP-7 is expressed in adult kidneys, its expression is frequently down regulated in the face of renal failure. Therefore, in vivo-produced BMP-7 can help restore levels of BMP-7 to normal physiological levels, leading to the control and regression of the fibrosis associated with tubulo-interstitial nephritis and CRF.

As used herein, a pharmaceutical composition according to the invention is said to have “therapeutic efficacy”, or to be “therapeutically effective”, if administration of that amount of the composition is sufficient to cause a significant improvement of the clinical signs or measurable markers of the disease in a mammalian subject suffering from ARF or CRF. As used herein, a pharmaceutical composition according to the invention is said to have “prophylactic efficacy” or to be “prophylactically effective”, if administration of that amount of the composition is sufficient prevent the development of ARF in a subject. The term “therapeutically effective” may also be used herein, in a more general sense, to refer to an amount of a composition that is either sufficient to cause a significant improvement of the clinical signs or measurable markers of disease in a mammalian subject suffering from ARF or CRF, or that is sufficient to prevent the development of ARF in a subject.

Measurable markers of renal function, which are also useful in evaluating the ARF or CRF status of a subject, are well known in the medical and veterinary literature and to those of skill in the art, and include, but are not limited to, blood urea nitrogen or “BUN” levels (both static measurements and measurements of rates of increase or decrease in BUN levels), serum creatinine levels (both static measurements and measurements of rates of increase or decrease in serum creatinine levels), measurements of the BUN/creatinine ratio (static measurements of measurements of the rate of change of the BUN/creatinine ratio), urine/plasma ratios for creatinine, urine/plasma ratios for urea, glomerular filtration rates (GFR), serum concentrations of sodium (Na+), urine osmolarity, daily urine output, and the like. Of the above, measurements of the plasma concentrations of creatinine and/or urea or BUN are particularly important and useful readouts of renal function.

Normal values for serum creatinine concentrations range from about 0.5 to about 1.6 mg/decilitre (“dl”) in dogs and from about 0.5 to about 1.9 mg/dl in cats. The upper limit of the normal physiological range of serum creatinine levels is slightly higher in cats than in dogs. With the exception of diet, factors influencing physiological values of serum creatinine concentration are poorly understood. It is known that a diet rich in protein has the potential to cause transient hypercreatinemia. For example, an increase of around 25% in serum creatinine concentration can occur over a 6-9 hour period when healthy dogs are fed with commercial food. The relevance of minor variations of creatinemia are difficult to interpret, and the smallest relevant variation between two successive measurements of creatinine levels is considered to be a change in concentration of 35 umol/l from normal values.

The upper limit of the normal physiological range for BUN levels in fasting dogs and cats ranges from about 8.8 to about 25.9 mg/dl in dogs, and from about 15.4 to about 31.2 mg/dl in cats—the upper limits of the normal range are slightly higher in cats than in dogs. BUN levels, like creatinine levels, are influenced by diet. Other factors that can lead to variation in BUN levels include long-term glucocorticoide treatment and/or hepatocellular failure.

Any significant increase of serum creatinine levels and/or BUN levels above their normal physiological ranges is a sign of a reduced ability of the kidneys to eliminate waste and catabolites (i.e., excretory failure).

Experimental demonstration of the efficacy of the methods and compositions of the present invention (e.g. the methods and compositions useful for gene therapy with BMP-7 or functional equivalents of BMP-7), can be performed in a variety of ways, for example, by demonstrating that subjects treated using the methods and compositions of the present invention exhibit a significantly reduced elevation of plasma creatinine and/or BUN, as compared to placebo-treated subjects, when exposed to a trigger or risk factor such as, for example, a toxicant (e.g., HgCl₂) or a procedure that induces renal ischemia (e.g., bilateral renal arteries occlusion).

Similarly, tissue readouts can be used to demonstrate the efficacy of the methods and compositions of the present invention. Examples of suitable tissular readouts include the quantification of tubulo-interstitial nephritic lesions (“TIN” lesions) within the cortical parenchyma of the kidney, and to a lesser extent, withing the medullary parenchyma of the kidney. It is well documented that renal interstitial fibrosis associated with tubulo-interstitial nephritis (TIN) is a common final pathway of kidney disorders with a wide spectrum of diverse etiologies. Deterioration of renal function is largely determined by the extent of the tubulo-interstitial lesions in many forms of renal diseases, and also in several experimental animal models. Accordingly, method or composition that is able to slow down or reverse the evolution of TIN fibrosis has the potential to benefit all kidney disorders through a disease-modifying mechanism (i.e., by limiting the degradation and disorganization of the structural elements of kidney tissues). Experimental demonstration of the efficacy of the BMP-7 gene therapy methods and compositions of the present invention can be demonstrated from the observation that BMP-7-treated subjects have significantly reduced tubulo-interstitial lesions in the kidneys than controls as assessed using the unilateral ureteral obstruction or “UUO” model. The UUO model is a well-established animal model of chronic progression of renal fibrosis associated with progressive tubular atrophy and interstitial collagen accumulation. The UUO model is well known in art, and the unilateral ureteral obstruction procedure can be readily performed by those of ordinary skill in the art. The UUO model is typically associated with very significant tubulo-interstitial pathology and with minimal glomerular lesions, and is a relevant and useful experimental model for demonstrating the efficacy of the methods and compositions of the present invention, for example the demonstrating the efficacy of the gene therapy strategy disclosed herein which is based on the in vivo expression of BMP-7 or functional equivalents of BMP-7. Using this model, the evaluation of TIN in the renal cortex can be determined using conventional hematoxylin and eosin (or “H&E”) staining and/or collagen-specific Masson Trichrome staining of fixed tissues. Characterization of the lesions is based on the extent of tubular dilatation, epithelial atrophy, and interstitial expansion with myofibroblast activation and matrix deposition. Additional investigations can be based on immunohistochemmistry and histomorphometry techniques using, for example, .alpha.-smooth muscle actin (“.alpha.-SMA”) specific antibodies to characterize and quantify the level of epithelial to mesenchyme transition (or “EMT”) in the tissue. Complementary immunohistochemical analysis can also be performed with antibodies specific for collagen I or for fibronectin. Quantification of cellular infiltration is an additional readout that can be used to characterize the lesions. Immunohistochemical analysis of the latter can be conducted using, for example, anti ED-1 or anti mac-1 antibodies that are specific for macrophages. Collectively, the results of the above readouts can be used to provide a grade for the lesion.

In addition to the above, any other suitable methods or readouts for studying kidney disease and/or kidney function, including any other suitable animal models, can also be used to demonstrate the efficacy of the methods and compositions of the present invention, and to determine what amount of such compositions, or what modes of administration, will be therapeutically or prophylactically effective.

In one aspect, the present invention related to a non-viral vector capable of expressing, in vivo in a host, an Osteogenic Protein-1 Bone Morphogenetic Protein-7 (OP-1/BMP-7) polypeptide, or a variant or a fragment thereof. As used herein “BMP-7 polypeptide” may be used to refer to pre-pro, pro or mature BMP-7 polypeptides, wherein the pro and mature BMP-7 polypeptides may be fused to a BMP-7, IGF-1 or tPA signal peptide. The BMP-7 polypeptides of the present invention maybe, e.g., of human, feline or canine origin. In one embodiment the vector contains and expresses in the host a pre-proBMP-7, a proBMP-7 or a mature BMP-7 nucleotide sequence or gene. The nucleotide sequence or gene encoding the pre-proBMP-7 polypeptide, the proBMP-7 polypeptide or the mature BMP-7 polypeptide originates from a mammal, for example a human, cat or a dog.

BMP-7 is also known as Osteogenic Protein-1 or “OP-1”, and is a member of the transforming growth factor-.beta. or “TGF-.beta.” superfamily. It is a secreted protein that is processed from the pro-protein to yield the carboxy-terminal mature protein. Within the mature protein there is a conserved pattern of seven cysteine residues. The active form of the protein is a disulfide-bonded homodimer. In its mature, native form, naturally occurring BMP-7 is a glycosylated dimer having an apparent molecular weight of about 30-36 kDa, as determined by SDS-polyacrylamide gel electrophoresis (“SDS-PAGE”). When reduced, the 30 kDa protein gives rise to two glycosylated polypeptide subunits having apparent molecular weights of about 16 kDa and 18 kDa. The unglycosylated protein has an apparent molecular weight of about 27 kDa. When reduced, the 27 kDa unglycosylated protein gives rise to two unglycosylated polypeptide chains, having molecular weights of about 14 kDa and 16 kDa.

Typically, the naturally occurring BMP-7 protein is translated as a precursor, having an N-terminal signal peptide sequence, a “pro” domain, and a “mature” protein domain. The signal peptide is 29 residues long and is cleaved off rapidly upon translation at a cleavage site that can be predicted using the method of Von Heijne (1986), Nucleic Acid Research, 14; 4683-4691. The “pro” domain has 264 residues in human, canine, swine and bovine BMP-7, and 263 residues in mouse BMP-7. The pro domain is cleaved to yield the “mature” C-terminal domain of 139 residues, which includes the conserved seven-cysteine C-terminal domain of 102 residues. As referred to herein, the “pro form” of the BMP-7 polypeptide refers to a protein comprising a pair of polypeptides, each comprising a pro domain in either covalent or non-covalent association with the mature domain of the BMP-7 polypeptide. The pro form appears to be the primary form secreted from cultured mammalian cells. The “mature form” of the protein refers to the mature C-terminal domain which is not associated, either covalently or non-covalently, with the pro domain.

As used herein the terms “pre-pro BMP-7”, “pro BMP-7”, “mature BMP-7” and “BMP-7 may include any and all of the known naturally occurring variants, of these proteins including, but not limited to, derivatives, mutants, homologues, orthologs, allelic variants, allelic polymorphs, polymorphic variants, phylogenetic counterparts, and also any and all non-naturally occurring variants of these proteins, including but not limited to derivatives, mutants, fragments, fusion proteins, and the like. As used herein, the term “variant” encompasses all such naturally occurring and non-naturally occurring variants. In particular, the present invention encompasses all such variants that retain the feature of being useful for the therapeutic or prophylactic treatment of renal diseases including ARF and CRF, and/or that retain BMP-7 activity.

These functionally equivalent variants, derivatives, and fragments, and the like display the ability to retain BMP-7 activity. A functional equivalent, as used herein, refers to any BMP-7 variants, derivatives, fragments, and the like that meet either of the following two criteria (a) they have a significant level of amino acid sequence homology with the protein sequence of BMP-7 as described herein, or is encoded by a nucleotide that has a significant level of nucleotide sequence homology with the protein sequence of BMP-7 as described herein; or (b) they have the ability to provide a statistically different response in the treated group as compared to a placebo treated group in at least one of the following experimental models of renal failure in rodents: (i) a toxicant-induced or ischemic-induced renal failure model, where reduced elevation of plasma creatinine or BUN is expected in the treated as compared to the control/placebo group; (ii) a UUO model of renal failure, where reduced lesion grading is expected in the treated group as compared to the control/placebo group.

By way of illustration of variants, derivatives, and the like that are encompassed by the present invention include, but are not limited to, BMP-7 variants, derivatives, and the like that are encoded by nucleotide sequences that are not exactly the same as the nucleotide sequences disclosed herein, but wherein the changes in the nucleotide sequences do not change the encoded amino acid sequence, or result in conservative substitutions of amino acid residues, deletion of addition of one or a few amino acids, substitution of amino acid residues by amino acid analogs that do not significantly affect the properties of the encoded polypeptides, and the like. Examples of conservative amino acid substitutions include glycine/alanine substitutions; valine/isoleucine/leucine substitutions; asparagine/glutamine substitutions; aspartic acid/glutamic acid substitutions; serine/threonine/methionine substitutions; lysine/arginine substitutions; and phenylalanine/tyrosine/tryptophan substitutions. Other types of substitutions, variations, additions, deletions and derivatives that result in functional BMP-7 derivatives, as described above, are also encompassed by the present invention, and one of skill in the art would readily know how to make, identify, or select such variants or derivatives, and how to test for BMP-7 activity of those variants or derivatives. One of skill in the art may optimize the expression of the BMP-7 polypeptides of the invention by removing cryptic splice sites, by adapting the codon usage by introducing a Kozak consensus sequence before the start codon, by changing the codon usage or combination thereof to improve expression.

In another embodiment, the present invention comprises a pre-proBMP-7 polypeptide variant having at least 90%, at least 91%, at least 92%, at least 93%, at least 94%, at least 95%, at least 96%, at least 97%, at least 98% or at least 99% homology or identity with its naturally occurring counterpart.

In another embodiment the invention comprises a mature BMP-7 polypeptide variant having at least 97%, at least 97.5%, at least 98%, at least 98.5%, or at least 99% homology or identity with its naturally occurring counterpart.

For the purposes of the present invention, sequence identity or homology is determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266, 460-480; Altschul et al., Journal of Molecular Biology 1990, 215, 403-410; Gish & States, Nature Genetics, 1993, 3: 266-272; Karlin & Altschul, 1993, Proc. Natl. Acad. Sci. USA 90, 5873-5877.

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

The terms “protein”, “polypeptide” and “polypeptide fragment” are used interchangeably herein to refer to polymers of amino acid residues of any length.

In certain embodiments, the expression vector comprises a polynucleotide that encodes a mature BMP-7 polypeptide, wherein the polypeptide is fused to a peptide signal sequence that is, or that comprises or is derived from the corresponding BMP-7 signal peptide. In other embodiments, the signal peptide sequence may be, or comprise or be derived from, other BMP-7 signal peptides.

The present invention further relates to vectors containing and expressing a polynucleotide encoding the proBMP-7 polypeptide, wherein the pre-BMP-7 signal peptide is deleted and wherein a peptide signal sequence from a different origin is fused to the proBMP-7 polypeptide. For example, in certain embodiments, the peptide signal sequence may be the insulin-like growth factor 1 (IGF-1) or the tissue plasminogen activator (tPA) peptide signal sequence.

In some embodiments, the present invention encompasses a vector capable of expressing pre-proBMP-7, proBMP-7, mature BMP-7, or a variant or fragment thereof. For the mature BMP-7 or the proBMP-7, it is preferred that the nucleotide sequence encoding the peptide is preceded immediately by a nucleotide sequence in-frame encoding a peptide signal in order to facilitate the secretion of BMP-7 into the extra cellular medium. The signal sequence can be the natural sequence from the pre-proBMP-7 or a peptide signal from a secreted protein e.g. the signal peptide from the tissue plasminogen activator protein (tPA), in particular the human tPA (S. Friezner Degen et al J. Biol. Chem. 1996, 261, 6972-6985; R. Rickles et al J. Biol. Chem. 1988, 263, 1563-1569; D. Berg. et al Biochem. Biophys. Res. Commun. 1991, 179, 1289-1296), or the signal peptide from the Insulin-like growth factor 1 (IGF1), in particular the equine IGF1 (K. Otte et al. Gen. Comp. Endocrinol. 1996, 102(1), 11-15), the canine IGF1 (P. Delafontaine et al. Gene 1993, 130, 305-306), the feline IGF1 (WO-A-03/022886), the bovine IGF1 (S. Lien et al. Mamm. Genome 2000, 11(10), 877-882), the porcine IGF1 (M. Muller et al. Nucleic Acids Res. 1990, 18(2), 364), the chicken IGF1 (Y. Kajimoto et al. Mol. Endocrinol. 1989, 3(12), 1907-1913), the turkey IGF1 (GenBank accession number AF074980). The signal peptide from IGF1 may be natural or optimized, in particular optimized by removing cryptic splice sites and/or by adapting the codon usage.

As used herein the term “polynucleotide” is used to refer to a polymeric form of nucleotides of any length, which contain deoxyribonucleotides or ribonucleotides.

The present invention further encompasses a vector containing and expressing a polynucleotide encoding a BMP-7 polypeptide operably linked to a promoter element and optionally also linked to an enhancer. The enhancers and/or promoters may be selected from among those promoters that are known in the art, and that are suitable for expression of BMP-7 in the plasmids of the present invention. Many such promoters are known in the art, and suitable promoters can readily be selected by those of skill in the art. For example, there are various cell and/or tissue specific promoters (e.g., muscle, endothelial cell, liver, somatic cell, and stem cell specific promoters), and various BMP-7 promoters, such as those isogenically specific for each animal species. For example, in one embodiment, if the human BMP-7 is to be expressed in human cells, the enhancers and/or promoters specific to corresponding human cells may be used in order to optimize expression of BMP-7 for the desired application.

In general, it is advantageous to employ a strong promoter functional in eukaryotic cells.

A strong cellular promoter that may be usefully employed in the practice of the invention is the promoter of a gene of the cytoskeleton, such as e.g. the desmin promoter (Kwissa M. et al., Vaccine, 2000, 18, 2337-2344), or the actin promoter (Miyazaki J. et al., Gene, 1989, 79, 269-277).

Functional sub fragments of these promoters, i.e., portions of these promoters that maintain an adequate promoting activity, are included within the present invention. A promoter in the practice of the invention consequently includes derivatives and sub-fragments of a full-length promoter that maintain an adequate promoting activity and hence function as a promoter, preferably promoting activity substantially similar to that of the actual or full-length promoter from which the derivative or sub-fragment is derived. Thus, a promoter in the practice of the invention can comprise or consist essentially of or consist of the promoter portion of the full-length promoter and/or the enhancer portion of the full-length promoter, as well as derivatives and sub-fragments.

The plasmids may comprise other expression control elements. It is particularly advantageous to incorporate stabilizing sequence(s), e.g., intron sequence(s), preferably the first intron of the hCMV-IE (PCT Application No. WO89/01036), the intron II of the rabbit .beta.-globin gene (van Ooyen et al., Science, 1979, 206, 337-344). As to the polyadenylation signal (polyA) for the plasmids, use can more be made of the poly(A) signal of the bovine growth hormone (bGH) gene (see U.S. Pat. No. 5,122,458), or the poly(A) signal of the rabbit .beta.-globin gene.

The term “plasmid”, as used herein, refers to a recombinant DNA or RNA plasmid that comprises a heterologous polynucleotide to be delivered to a target cell, such as in vivo. The heterologous polynucleotide may comprise a sequence of interest for purposes of therapy, and may optionally be in the form of an expression cassette. As used herein, a “plasmid” need not be capable of replication in the ultimate target cell or subject.

The term “recombinant” as used herein means a polynucleotide semisynthetic, or synthetic origin, which either does not occur in nature or is linked to another polynucleotide in an arrangement not found in nature.

The term “heterologous” as used herein derived from a genetically distinct entity from the rest of the entity to which it is being compared. For example, a polynucleotide may be placed by genetic engineering techniques into a plasmid derived from a different source, and is thus a heterologous polynucleotide. A promoter removed from its native coding sequence and operatively linked to a coding sequence other than the native sequence is accordingly a heterologous promoter.

The polynucleotides of the invention may comprise additional sequences, such as additional coding sequences within the same transcription unit, controlling elements such as promoters, ribosome binding sites, transcription terminators, polyadenylation sites, additional transcription units under control of the same or different promoters, sequences that permit cloning, expression, homologous recombination, and transformation of a host cell, and any such construct as may be desirable to provide embodiments of this invention.

In one embodiment, elements for the expression of BMP-7 are present in an inventive plasmid. In one embodiment, this comprises, consists essentially of, or consists of an initiation codon (ATG), a stop codon and a promoter, and optionally also a polyadenylation sequence for certain plasmids. When the polynucleotide encodes a polypeptide fragment, e.g. BMP-7, advantageously, in the vector, an ATG is placed at 5′ of the reading frame and a stop codon is placed at 3′. Other elements for controlling expression may be present, such as enhancer sequences, stabilizing sequences, such as intron and signal sequences permitting the secretion of the protein.

According to one embodiment of the invention, the expression vector is a plasmid vector or a DNA plasmid vector, in particular an in vivo expression vector.

The term plasmid covers any DNA transcription unit comprising a polynucleotide according to the invention and the elements necessary for its in vivo expression in a cell or cells of the desired host or target; and, in this regard, it is noted that a supercoiled or non-supercoiled, circular plasmid, as well as a linear form, are intended to be within the scope of the invention. Each plasmid may comprise or contain or consist essentially of, in addition to the polynucleotide encoding the pre-proBMP-7, the proBMP-7 or the mature BMP-7 polypeptide, the BMP-7 polypeptide being preferably from, e.g., human, dog or cat origin, variant, analog or fragment, operably linked to a promoter or under the control of a promoter or dependent upon a promoter.

The present invention also relates to a pharmaceutical composition comprising genetic material expressing in vivo under appropriate or suitable conditions or in a suitable host cell. The pharmaceutical compositions can comprise, consist essentially of, or consist of one or more plasmids, e.g., expression vectors, such as in vivo expression vectors, comprising, consisting essentially or consisting of and expressing one or more polynucleotides encoding a BMP-7 polypeptide, optionally fused with a BMP-7, IGF-1 or tPA signal peptide, in a pharmaceutically or veterinarily acceptable carrier, excipient or vehicle. Advantageously, the plasmid contains, consists essentially of, or consists of and expresses at least one polynucleotide encoding a human, dog or cat BMP-7 polypeptide, optionally fused with a BMP-7, IGF-1 or tPA signal peptide, in a pharmaceutically or veterinarily acceptable carrier, excipient or vehicle. Thus, according to an embodiment of the invention, the other genetic material in the composition comprises a polynucleotide that encodes, and under appropriate circumstances expresses one or more other proteins, polypeptides or peptides than the BMP-7 polypeptide.

In an advantageous embodiment, the invention provides for the administration of a therapeutically effective amount of a formulation for the delivery and expression of a BMP-7 polypeptide in a target cell. Determination of the therapeutically effective amount is routine experimentation for one of ordinary skill in the art. In one embodiment, the formulation comprises a non-viral vector comprising a plasmid comprising a polynucleotide that expresses BMP-7 polypeptide and a pharmaceutically or veterinarily acceptable carrier, vehicle or excipient. In an advantageous embodiment, the pharmaceutically or veterinarily acceptable carrier, vehicle or excipient facilitates transfection and/or improves preservation of the non-viral vector and/or plasmid.

The pharmaceutically or veterinarily acceptable carriers or vehicles or excipients are well known to the one skilled in the art. For example, a pharmaceutically or veterinarily acceptable carrier or vehicle or excipient can be water or a 0.9% NaCl (e.g., saline) solution or a phosphate buffer. Other pharmaceutically or veterinarily acceptable carrier or vehicle or excipients that can be used for methods of this invention include, but are not limited to, poly(L-glutamate) or polyvinylpyrrolidone. The pharmaceutically or veterinarily acceptable carrier or vehicle or excipients may be any compound or combination of compounds facilitating the administration of the non-viral vector, increasing the level of expression or increasing the duration of expression. Doses and dose volumes are herein discussed in the general description and can also be determined by the skilled artisan from this disclosure read in conjunction with the knowledge in the art, without any undue experimentation.

In one embodiment, the pharmaceutical composition is directly administered in vivo, and the encoded product is expressed by the vector in the host. The in vivo delivery of a non-viral vector comprising a plasmid encoding and expressing the BMP-7 described herein can be accomplished by one of ordinary skill in the art given the teachings of the above-mentioned references.

In the case of therapeutic and/or pharmaceutical compositions based on a plasmid, a dose can comprise, consist essentially of or consist of, in general terms, about in 1 ug to about 2000 ug, advantageously about 50 ug to about 1000 ug and more advantageously from about 100 ug to about 800 ug of plasmid expressing an OP-1/BMP-7 polypeptide.

The pharmaceutical composition can be administered by vascular delivery. The pharmacodynamics-based plasmid DNA gene delivery method based on the change of the hydrodynamics of blood circulation in the recipient animals following the injection of a large volume of DNA solution within a short period of time. It has been demonstrated that the delivery of naked DNA through intraportal or intrahepatic vein injection result in high level of gene expression. The specific expression in the mammalian kidney can be achieved following direct injection into the inferior vena cava (IVC). Through this procedure, expression in the kidney was 10- to 1000-fold higher than in other organs.

The dose volumes can be between about 0.1 and about 2 ml, advantageously between about 0.2 and about 1 ml.

The present invention contemplates at least one administration to an animal of an efficient amount of the therapeutic composition made according to the invention. The animal may be male, female, pregnant female and newborn. This administration may be via various routes including, but not limited to, intramuscular (IM), subcutaneous (SC), intravascular (IV) or intrarenal injection. Alternative routes to reach the kidneys are: renal artery, injection into the renal subcapsular space, retrograde injection from the ureter or parenchymal injection.

It should be understood by one of skill in the art that the disclosure herein regarding administration of the compositions of the invention is provided by way of example, and that the present invention is not limited to the specific examples described. From the disclosure herein, and from the knowledge in the art, the skilled artisan can determine the number of administrations, the administration route, and the doses to be used for each administration of the compositions of the present invention without any undue experimentation.

In a preferred embodiment, the present invention relates to the use of, and to compositions comprising, a non-viral vector comprising a plasmid encoding and capable of expressing, a pre-proBMP-7, a proBMP-7, a BMP-7 mature polypeptide, or a variant, derivative or fragment thereof, for the treatment and/or prevention of ARF, CRF. Or other kidney conditions.

In one embodiment the invention relates to the use of the pharmaceutical compositions according to the present invention to treat mammals presenting an increase in their serum creatinine concentration and/or an increase in their BUN concentration, or an increase in their urine specific gravity.

The invention will now be further described by way of the following non-limiting examples.

### EXAMPLE 1

The objective of the present study was to prepare chitosan nanoparticles incorporating a relatively large plasmid encoding for osteogenic protein (OP)-1 and to determine the ability of these nanoparticles to transfect adult cells such as canine articular chondrocytes in vitro. The positive charge of chitosan acted to condense the relatively large negatively-charged OP-1 plasmid such that it could be incorporated into nanoparticles. Incorporation of the plasmid into the chitosan nanoparticles did not affect the structural integrity of the plasmid as demonstrated by gel electrophoresis. The morphology and size of the nanoparticles were found to vary with the chitosan:plasmid weight ratio. Nanoparticles formulated with a chitosan:plasmid ratio of 10:1 were of uniformly small size (less than 250 nm) and spherical shape. These nanoparticles had a positive charge of about 20 mV. FITC-labeled chitosan nanoparticles were found in virtually all of the cells after 24 hours of incubation with the nanoparticles, and confocal microscopy revealed FITC-related fluorescence in the nucleus of the chondrocytes. Although transfection of the chondrocytes was demonstrated by the fluorescence of cells treated with chitosan nanoparticles containing the plasmid for the enhanced green fluorescence protein, cells transfected with nanoparticles incorporating the larger OP-1 plasmid did not show OP-1 expression measured by ELISA for up to 2 weeks in culture. These results indicate that although a large plasmid can be successfully incorporated within chitosan nanoparticles, the size of the plasmid incorporated within the nanoparticles may still significantly affect gene transfer to cells.

### Introduction

It is now well established that growth factors may play an important therapeutic role in the treatment of cartilage defects. However, growth factors are subject to clinical limitations that include a short half-life in vivo requiring multi-dose administration and expense for the large quantities that may be required. These potential problems have stimulated interest in the implementation of gene transfer methods to enable the local, sustained expression of the growth factors. While high transfection efficiencies can be readily achieved with viral vectors, the potential for untoward biological responses to the viral vector has prompted investigation of non-viral transfection approaches. In contrast, the non-viral vectors may provide several advantages such as non-infectivity, absence of immunogenicity, the possibility of multi-dose administration, and low cost.

Non-viral vectors including cationic liposomes, cationic lipids, and synthetic and natural polymers have also been employed as delivery vehicles for genes. The advantage of cationic lipids, such as Lipofectamine, is that they have a high transfection efficiency in vitro. However, there biocompatibility profile in vivo has not yet been established. One of the natural polymers employed as a non-viral vector is chitosan. Chitosan is a naturally occurring polysaccharide comprising two subunits, D-glucosamine and N-acetyl-D-glucosamine linked together by M(1,4) glycosidic bonds. Its desirable attributes as a non-viral vector include: a positive charge capable of condensing DNA with which it interacts and favoring interactions with the negative charge of the cell membrane; general biocompatibility; and ability to form nanometer and micrometer sized complexes with DNA. Moreover, chitosan-DNA nanoparticles did not induce release of proinflammatory cytokines from macrophages. A host of other advantages of employing chitosan as a delivery vehicle have been proposed including: (1) conjugation of other molecules to the chitosan to interact with specific membrane receptors, including integrins, that could elicit selected cell behavior including receptor mediated endocytosis; (2) incorporation of agents to inhibit the intracellular degradation of the plasmid; and (3) inclusion of other biologically active substances. Of interest is that chitosan was found to provide better transfection than cationic liposome under certain conditions; whereas cationic liposome (lipofectin)-associated gene expression was inhibited by serum, chitosan showed resistance to serum.

The objective of the present study was to prepare chitosan nanoparticles incorporating a relatively small plasmid containing the gene encoding for the enhanced green fluorescent protein (EGFP) and/or a relatively large plasmid containing the gene for osteogenic protein (OP)-1 [also known as bone morphogenetic protein (BMP)-7], and to evaluate the ability to use these chitosan nanoparticles to transfect adult articular chondrocytes. Most of the non-viral strategies directed toward the transfection of chondrocytes for cartilage repair have been lipid or liposome-based methods. The reason for selecting OP-1 for this investigation is that several studies have demonstrated the favorable effects of OP-1 on chondrogenesis in vitro and on cartilage repair in vivo. While there have been several reports of OP-1 gene transfer to cells using viral vectors, there have been only 2 reports of nonviral tranfection of cells with OP-1 plasmid DNA, and no studies investigating the use of nanoparticles as delivery vehicles for the gene. The relatively large size of the OP-1 plasmid used in this study (˜9-10 kb) compared to the plasmid size for EGFP (˜4.7 kb) gave a good comparison to determine if plasmid size has an effect on transfectibility using chitosan nanoparticles.

The emphasis in the current study was the methodology for preparing nanoparticles incorporating a relatively large plasmid, and the effects of the conditions under which the nanoparticles were produced on their characteristics (viz., size and shape). One of the specific aims of the study was to determine how the chitosan:plamid OP-1 (pOP-1) ratio affected the size, morphology, and charge of the nanoparticles. A second aim of the study was to assess the structural integrity of the OP-1 complexed with chitosan using gel electrophoresis. The third aim of the study was to demonstrate by fluorescence microscopy and ELISA the functionality of the chitosan nanoparticles encoding an enhanced green fluorescence protein (EGFP) and pOP-1, respectively, using adult articular chondrocytes. There has been only one prior report of the uptake of nanoparticles by chondrocytes incorporating the relatively small plasmid for EGFP (pEGFP). This study investigates the incorporation and characterization of a plasmid much larger than pEGFP into chitosan nanoparticles and evaluates its effectiveness in transfecting chondrocytes in vitro.

### Materials and Methods

Preparation of Chitosan Nanoparticles containing pOP-1 and a Fluorescent Label

Chitosan stock solution (0.2%, w/v) was made as follows. Ten milligrams of medium to high molecular weight chitosan (Cat. # 41,796-3, Sigma-Aldrich, Inc., St. Louis, Mo.) was added to a tube containing 11.6 μl acetic acid in 4.0 ml water and then kept at 37° C. overnight. After the chitosan was dissolved, 0.028 gm of sodium acetate (Fisher Scientific, Fair Lawn, N.J.) was added to the tube, the pH was adjusted to 5.5, and the volume was increased to 5 ml. Chitosan working solutions with different concentrations were made from the stock by dilution with 5 mM acetate buffer (pH 5.5) and sterile filtered.

The plasmid OP-1 (pW24, Cell & Molecular Technologies, Inc, Phillipsburg, N.J.) working solution (200 μg/ml) was prepared with filtered 5 mM sodium sulfate (Fisher Scientific). The size of the plasmid, estimated by Cell & Molecular Technologies based on 3 restriction enzyme preparations was: 10,346 by (HindIII restriction enzyme), 9,406 by (PstI) and 7,793 by (EcoRI). A complex coacervation method previously described was used to make chitosan nanoparticles complexing the pOP-1. Complex coacervation is the separation caused by the interaction of two oppositely charged colloids. Briefly, 100-150 μl chitosan and pOP-1 solutions were heated separately at 55° C. for 30-45 min. Equal volumes of both solutions were quickly mixed together and vortexed for 30-45 sec. The nanoparticles were used without further purification.

Fluorescein-5-isothiocyanate (FITC) was added to the nanoparticles along with the OP-1 plasmid using the following procedure. Forty milligrams FITC (Sigma-Aldrich, Inc.) was dissolved in 5 ml dimethylsulfoxide, 0.5 ml chitosan (0.1%) solution was added, and the mixture was stirred for 1-2 hr. The FITC-labeled chitosan was then precipitated with 25 ml NaOH solution at pH 8. After centrifugation of the precipitated product at 4800 rpm for 15 min. the supernatant solution was discarded. The pellet was washed three times with 25 ml distilled water. The resulting chitosan was resuspended in acetate buffer and then used to prepare nanoparticles incorporating pOP-1, as described above.

The plasmid encoding for EGFP (Clontech, Mountain View, Calif.), with a plasmid size of 4.7 kb, was used as a reporter gene to transfect chondrocytes. The plasmid was amplified in Escherichia coli host strain DH5a, and purified using the QIAfilter plasmid Mega kit (QIAGEN Inc.—USA, Valencia, Calif.). The EGFP and OP-1 plasmids were incorporated into the same chitosan nanoparticle (chitosan:total plasmid weight ratio of 10:1 and a pEGFP:pOP-1 weight ratio of 7:3) in order to track the transfection of the chondrocytes by the chitosan nanoparticles via fluorescence. Realizing that plasmid size may have an effect on gene transfer and that GFP expression alone would not verify that the OP-1 plasmid was incorporated or expressed by the transfected chondrocytes, chitosan nanoparticles incorporating OP-1 plasmid alone were similarly synthesized and used for transfection in order to directly assess the over-expression of the larger OP-1 plasmid by ELISA.

### Electrophoresis of Nanoparticles and Plasmid Stability

Incorporation of the pOP-1 into chitosan nanoparticles was studied using agarose gel electrophoresis. Two issues were addressed through experimentation: the retention of the plasmid in chitosan nanoparticles prepared with various amounts of the plasmid, and the structure of the plasmid after being released by the chitosan. Plasmid integrity of the plasmid released from the nanoparticles was determined using restriction enzymes. For each gel, naked pOP-1 (0.8 μg in 80 μl 5 mM sodium sulfate solution) was used as the control. A 1 kb DNA Ladder (Cat. No. 10381-010, Invitrogen, Carlsbad, C) was also run on each gel in parallel.

Increasing amounts of chitosan were mixed with a single quantity of pOP-1 (10 ug), to yield the following weight ratios of chitosan to pOP-1: 0.5:1; 1:1; 2:1; 5:1; 10:1; 20:1; and 40:1. The nanoparticles were loaded onto a 0.8% agarose gel in Tris-borate-ethylenediamine tetraacetic acid, EDTA (TBE) buffer (×1) with ethidium bromide. The gel was run at 100v for 60-80 min, and then photographed with Foto/Analyst Visionary (Fotodyne Inc., New Berlin, Wis.).

The structural integrity of pOP-1 released from the nanoparticles was also investigated by electrophoresis. To digest the chitosan, 80 μl of the nanoparticle suspensions of the various chitosan:pOP-1 ratios equivalent to 0.8 μg pOP-1 was added to 8 μl chitosanase (0.24 U/ml, Sigma-Aldrich, Inc.) and 20 μl lysozyme (100U/ml, Sigma-Aldrich, Inc.) in 5 mM sodium acetate buffer (pH 5.5). The mixture was incubated at 37° C. for 4 hr. The digested solutions were then run on the gel.

Samples of the stock pOP-1 solution and aliquots (16 μl) of the digested solutions from each of the nanoparticle-pOP-1 preparations were cut with Hind (III) restriction endonuclease (New England BioLabs, Ipswich, Mass.) and run on gels. Samples of the stock plasmid were also cut with SaII, XhoI BamHI and EcoRI restriction enzymes, and analyzed by gel electrophoresis as described above, in order to estimate the size of the plasmid.

### Environmental Scanning Electron Microscopy

Environmental scanning electron microscopy (ESEM, XL30, FEI/Philips, Hillsboro, Oreg.) was used to investigate the size and shape of the nanoparticles. Samples were prepared by placing 1 μl of the nanoparticle suspension onto a slide and air drying the preparation. The air-dried samples were then observed directly under ESEM, without the need to apply a conductive coating. Nanoparticles with chitosan:plasmid weight ratios of 5:1, 10:1, and 20:1 were examined by ESEM.

Determination of Nanoparticle Size Distribution

The particle size distribution was determined by the dynamic light scattering technique performed at 25° C. with a Brookhaven 200SM goniometer, a BI-9000AT digital auto-correlator, and Spectra-Physics Argon laser operating at 514 nm (Brookhaven Instruments Corporation, Holtsville, N.Y.). The measured scattering intensities were analyzed by the software provided by Brookhaven Instruments Corporation. Nanoparticles with chitosan: plasmid weight ratios of 5:1, 10:1, and 20:1 were evaluated for their particle size distribution.

Zeta Potential

The zeta potential of the nanoparticles, reflecting their charge, was measured with Brookhaven's Zeta Plus apparatus (Brookhaven Instruments Corporation). The electrophoretic mobility of nanoparticles, with chitosan:plasmid weight ratios of 5:1, 10:1, and 20:1, in acetate buffer (pH 5.5) was determined at 25° C. A pH 5.5 was used because this was the pH at which the nanoparticles were made. The zeta potential was calculated using the Hückel approximation for small particles in low dielectric constant medium.

### Chondrocyte Uptake of Nanoparticles Incorporating FITC and Plasmids for Enhanced Green Fluorescent Protein and OP-1

Chondrocytes were obtained by enzymatic digestion of the articular cartilage obtained from the knee joint of one adult dog. The cells were expanded in number in monolayer culture using a modification of the medium previously reported, consisting of Dulbecco's modified Eagle's medium, DMEM (4.5%, without L-Glutamine and with 1 mM Sodium Pyruvate), 0.1 mM nonessential amino acids, 10 mM N-2-Hydroxyehtylpiperazine-N′-2-ethanesulfonic (HEPES) buffer, 100 U/mL penicillin, 100 ug/mL streptomycin glutamate, 10% FBS (Invitrogen Corporation, Carlsbad, Calif.), and a mixture of the following growth factors (R&D Systems, Minneapolis, Minn.): TGF-31 (1 ng/ml), FGF-2 (5 ng/ml) and PDGF-bb (10 ng/ml). Chondrocytes subcultured once (passage 1) were used in this investigation.

Twenty-four hours prior to addition of the nanoparticles to the cultures, the cells were seeded onto glass-bottomed dishes (14-mm diameter wells; MatTek Corporation, Ashland, Mass.) at a density of 5×10⁴ cells per dish. The cells were cultured in the medium described above. At 80-90% confluence, the medium was removed and replaced with a 250-μl suspension of fluorescence-labeled nanoparticles in serum-free medium consisting of high glucose DMEM (4.5%, without L-Glutamine and with 1 mM Sodium Pyruvate), 0.1 mM nonessential amino acids, 10mM HEPES buffer, 100 U/mL penicillin, 100 ug/mL streptomycin glutamate, ITS⁺¹(100×, by Sigma Chemical, St. Louis, MO), 0.1 mM ascorbic acid 2-phosphate, 1.25 mg/ml bovine serum albumin, 10 ng/mL of TGF-β1, and 100 nM dexamethasone. Four hours later, another 250 μl of serum-free medium was added. After an additional 20-hour incubation period, the chondrocytes were rinsed with phosphate-buffered saline (PBS) and 500 μl of serum-free medium added. For control experiments, nanoparticles without FITC and an FITC solution alone were used. Nanoparticle uptake by the chondrocytes was examined by fluorescence microscopy and confocal laser scanning microscopy.

Chondrocytes were also cultured with the nanoparticles which contained the EGFP and OP-1 plasmids together in the same nanoparticle or with nanoparticles incorporating OP-1 plasmid alone using the same culture conditions as described above. In control cultures, EGFP plasmid alone (i.e., not incorporated into nanoparticles) or combined with a lipid transfection reagent (GenePorter®; Gene Therapy Systems, Inc., San Diego, Calif.) was added directly to the cultures. Transfected cells were examined by transmitted fluorescence microscopy.

For cultures transfected with OP-1 plasmid nanoparticles, media were collected 1, 3, 5, and 7 days after transfection and assessed for OP-1 protein using a DuoSet human ELISA kit (R&D Systems, Minneapolis, Minn.). For a positive control group, chondrocytes were transfected with OP-1 plasmid using the GenePorter transfection reagent according to the manufacturer's instructions. For a negative control group, cultures were not treated with any transfection reagent or plasmid.

### Results

Electrophoresis of the OP-1 Plasmid Released from the Nanoparticles

The naked OP-1 plasmid in the stock solution was found to move slowly in the electrophoretic gel, likely due to the coiled configuration. After the digestion with restriction enzymes, the size of plasmid in the stock solution was estimated by summing the sizes of the prominent linear fragments: 14,500 by (XhoI); 13,800 by (BamHI); 12,800 by (EcoRI); 12,200 by (SaII); and 11,500 by [Hind (III)]. The size of the plasmid released from the digestion of the nanoparticles was estimated to be about 11,500 by using the Hind (III) enzyme.

The interaction between chitosan and pOP-1 was evaluated by agarose gel electrophoresis. At the lowest weight ratio of chitosan to pOP-1 of 0.5:1, some of the pOP-1 was released from the nanoparticles and migrated into the gel as demonstrated by a faint band corresponding to the location of the naked plasmid control. With an increasing chitosan:plasmid ratio, there was no sign of migration of the pOP-1 plasmid, indicating that it remained within the nanoparticles. A 1:1 ratio of chitosan to pOP-1 appeared to be sufficient to retain most of the plasmid in the polymer nanoparticles.

The electrophoretic mobility of the plasmid released by enzymatically digesting the nanoparticles was comparable to the stock plasmid and the stock pOP-1 treated with the digestion enzymes. This indicated that incorporation of the plasmid into the chitosan nanoparticles, and its release by enzymatic digestion of the chitosan, did not alter the structural integrity of the plasmid. There was no noticeable difference in the location of the bands from the released plasmid when compared to the stock control plasmids.

After Hind (III) restriction enzyme cleavage of the plasmid recovered from enzymatic digestion of the nanoparticles, the molecular profileswere found to be similar to that of the original pOP-1.

### Morphology of the Chitosan-pOP-1 Nanoparticles

ESEM revealed various morphologies of the nanoparticles prepared with chitosan:plasmid weight ratios of 5:1, 10:1, and 20:1. The nanoparticles with the chitosan:plasmid ratio of 10:1 were generally uniform in size and less than 500 nm; this small size and low atomic number precluded higher magnification imaging of the nanoparticles in the ESEM. Also of interest was the fact that the small particles did not display a tendency to aggregate. In contrast, nanoparticles prepared with the lower chitosan:plasmid weight ratio of 5:1 demonstrated varied morphologies with some spherical particles approximately twice the diameter of the 10:1 particles and other nanoparticles with a fibrous and branching structure, about 500 nm in width and up to 10 μm long. Nanoparticles prepared with the higher chitosan:plasmid weight ratio of 20:1 also displayed a fibrous and branching structure but of a substantially smaller size than the 5:1 particles, with the length of the features being about 1 μm. In the case of the 20:1 particles the branching morphology may have been the result of aggregation of elongated or fibrous particles.

### Particle Size Distribution

Dynamic light scattering revealed that the chitosan-OP-1 nanoparticles were polydisperse in size, with widely varying ranges of diameter based on the chitosan:pOP-1 ratio. The smallest average particle diameter and greatest percentage of nanoparticles of smallest diameter were recorded for the chitosan:pOP-1 nanoparticles with the 10:1 ratio. This was consistent with the ESEM findings. Nanoparticles prepared with a chitosan:pOP-1 ratio of 10:1 displayed an average diameter of 240 nm with a range of 25-613 nm. The preparation with the chitosan:pOP-1 ratio of 20:1, however, also appeared to comprise nanoparticles of comparably small size, with an average of 325 nm and a range of 18-562 nm. This would suggest that the larger branched particles seen in ESEM were aggregate of smaller sized particles. As suggested by ESEM, the light scattering results demonstrated that the particles prepared with a chitosan:pOP-1 ratio of 5:1 were twice the diameter of the nanoparticles with a ratio of 10:1. Moreover, light scattering showed that the range of diameters of the nanoparticles with the 5:1 ratio (114-2553 nm), was substantially higher than the range for the 10:1 nanoparticles.

### Charge of the Nanoparticles

The zeta potential of original chitosan solution (pH 5.5) was 45.5 ±1.1 mV. When the chitosan was coupled with the negatively charged pOP-1, the surface charge decreased to approximately one-half this value. Interestingly, with the increasing chitosan-plasmid weight ratio, the surface charge of the nanoparticles did not change much; the three nanoparticle preparations decreased to about 20 mV.

### Chondrocyte Uptake of Nanoparticles

Based on an average nanoparticle diameter and a chitosan density of about 0.3 gm/ml, we roughly estimated that the number of nanoparticles added to the chondrocyte monolayer was on the order of 10. Assuming that the cultures to which the nanoparticles were added contained approximately 100,000 cells, the number of nanoparticles per cell was estimated to be 100,000. The chondrocyte uptake of nanoparticles containing both OP-1 plasmid and FITC was observed by fluorescence microscopy using FITC-labeled chitosan-plasmid nanoparticles; as to be expected, no fluorescence was detected in the cultures in which the chitosan nanoparticles alone (without incorporation of FITC) were administered to the cells. The fluorescence images of the experimental cultures in which the FITC-labeled nanoparticles were used, demonstrated that the nanoparticles had been endocytosed by virtually all of the chondrocytes after 24 hours of incubation of the cells with the nanoparticles. This was demonstrated in the microscope by switching between the visible light and ultraviolet sources, in order to view all of the cells and then those fluorescing. In contrast the fluorescence from the chondrocytes treated with FITC solution alone appeared to come principally from the surface of the cells.

Confocal laser scanning microscopy was used to more clearly observe the intracellular distribution of the chitosan nanoparticles incorporating FITC. Confocal microscopy was necessary to demonstrate that the FITC-labeled nanoparticles were in the cell and not merely adsorbed onto the cell membrane. This could not be concluded from fluorescence microscopy. The confocal images showed that the FITC was distributed throughout the cytoplasm, and was also taken up into the nucleus of the chondrocytes.

Control cultures to which the EGFP plasmid was added as a solution (i.e., not incorporated into nanoparticles) did not display fluorescence. In contrast, the results showed EGFP expression in chondrocytes that were exposed to the nanoparticles which contained the EGFP and OP-1 plasmids in the same nanoparticle, indicating the capability of the chitosan nanoparticles to deliver the genes to the cells. Fluorescence microscopy revealed expression of EGFP in chondrocytes after 84 hrs of transfection. Although EGFP expression was achieved using the chitosan nanoparticles as the gene delivery vehicle, chondrocytes transfected with nanoparticles incorporating OP-1 plasmid alone did not show overexpression of OP-1 (by assaying for the protein in the media by ELISA) up to one week of culture. The positive controls (cells transfected with the OP-1 plasmid delivered by the GenePorter transfection reagent) displayed OP-1 in the media at Day 1 (13.4±3.3 ng/ml, n=3; mean±std. dev.) and on Day 3 (6.1±1.4 ng/ml, n =4), but not in any of the other collection time points (5 and 7 days of culture). Based on the negative controls, there was no constitutive expression of OP-1 by these cells in monolayer (i.e., no OP-1 released by chondrocytes not treated with any transfection reagent or plasmid).

### Discussion

The OP-1 plasmid employed in the current study was of relatively large size, estimated to be 12-14 kb. The plasmid has been employed in the commercial production of recombinant human OP-1 by Stryker Biotech (Hopkinton, Mass.). The reason for the specific structure of the plasmid and the requirements for its large size were outside the scope of the project. A recent study investigating co-transfection of rat calvarial cells with the genes for OP-1 and insulin-like growth factor (IGF)-1, used a modified form of the pW24 OP-1 plasmid that we used in the current work. In the prior experiment pW24 was digested with the restriction enzyme, Xho1. The OP-1 coding sequence was purified on agarose gels and re-ligated with the cytomegalovirus (CMV) promoter to produce a plasmid, that itself was rather large at 9.1 kb. That genes for critically important growth factors such as OP-1 may be of certain value in plasmids of large size compels the investigation of methodology for incorporating large plasmids into nanoparticles, which themselves may offer unique benefits for selected applications.

It was, therefore, of note in the current study that a plasmid of such a large size could be incorporated into nanoparticles. The advantage of chitosan for this application is that the positive charge of chitosan acted to condense the large negatively charged OP-1 plasmid such that it could be incorporated into nanoparticles. The OP-1 plasmid may be one of the largest plasmids that has ever been coupled with chitosan nanoparticles.

In general, migration of plasmid DNA on a gel can be retarded by the charge and/or molecular configuration and/or formation of complexes. When the stock plasmid was cleaved by restriction enzymes, the linear fragments displayed a characteristic electrophoretic profile. The avidity with which the pOP-1 was incorporated into the chitosan nanoparticles was demonstrated by the fact that free plasmid was evident on the electrophoretic gel only for the nanoparticles formulated with the lowest chitosan-plasmid ratio of 0.5:1. In nanoparticles with higher contents of chitosan, the polymer may have acted to condense the negative plasmid to a greater extent through electrostatic interactions thus resulting in higher affinity incorporation. A chitosan:plasmid weight ratio of at least 1:1 was necessary to complex the plasmid completely. Owing to the large size of the plasmid, this higher amount of chitosan may have been required to condense the pOP-1 enough to form nanoparticles.

Of importance is the finding that incorporation of pOP-1 into nanoparticles with all of the chitosan-plasmid ratios did not affect the structure of the plasmid. The electrophoretic migration of pOP-1 (1) in the digests from the enzymatic breakdown of the nanoparticles, and (2) in the restriction enzyme treated digests, matched the respective pOP-1 controls. These findings indicate that controlled release of pOP-1 from chitosan nanoparticles, that can be regulated, in part, by the chitosan:plasmid ratio, can be accomplished without affecting plasmid integrity, although further studies were still necessary to directly determine the functionality of the plasmid by assessing OP-1 protein expression. It will be interesting in future work to investigate the optimal conditions for preparing nanoparticles for incorporation of plasmids of varying size.

In this study, varying the chitosan:plasmid ratios had a noticeable effect on the morphology and size of the pOP-1 incorporated nanoparticles. The chitosan:plasmid mass ratio has been investigated by various groups as a important factor in the formulation of chitosan nanoparticles. Prior work has varied the chitosan:plasmid value from 2:1 to 5:1, and from 0.05:1 to 2.5:1 in experiments incorporating the same plasmid. In the present work, nanoparticles formulated with a chitosan:plasmid ratio of 10:1 were of uniformly small size (less than 250 nm) and spherical shape. This diameter was larger than that of earlier reports of 50-100 nm of chitosan nanoparticles containing the marker gene luciferase. This size difference may be due to the bigger size of the OP-1 plasmid. However, we also found in the present study, that the size and morphology, and apparent state of aggregation, varied dramatically with increased or decreased chitosan:plasmid ratio.

The small uniform spherical nanoparticles prepared with a chitosan:plasmid ratio of 10:1 had a positive charge, which may facilitate the binding of the nanoparticles to the negatively charged cells. The value of the positive charge was comparable to that recorded (23-24 mV at pH 5.0) for plasmid (luciferase)-chitosan complexes, several micrometers in diameter. In the present study the positive charge of the nanoparticles did not change much even with increasing chitosan-plasmid weight ratio. This result was in accordance with the former published data, which exhibited a zeta potential plateau in the presence of excess chitosan. Future work will be required to determine how the size, morphology and charge affect interactions with cells and performance of the nanoparticles as delivery vehicles of plasmid.

One of the findings of interest was the ability of the chitosan nanoparticles to gain entry into adult canine articular chondrocytes. This was demonstrated using chitosan nanoparticles incorporating FITC in addition to the OP-1 plasmid, confirming a recent study incubating rabbit chondrocytes with chitosan particles. The FITC-labeled chitosan nanoparticles, which also contained the OP-1 plasmid, were found in virtually all of the cells after 24 hours of incubation with the nanoparticles. The fluorescence appearance of the chondrocytes containing the FITC-labeled chitosan nanoparticles was similar to the fluorescence microscopy of phagocytic macrophages engulfing chitosan-FITC nanoparticles and tumor cell uptake of FITC-labeled chitosan microspheres. Transmission electron microscopy studies demonstrated the mechanism of uptake of chitosan-DNA nanoparticles by HeLa human cervix epitheliod cells to be spontaneous endocytosis with the nanoparticles being collected in invaginations of the plasma membrane. Once inside the cytoplasm the nanoparticles were found in small vesicles and large endosomal compartments. Of importance was the finding that other cell types (tumor cells) did not similarly endocytose the same chitosan-DNA nanoparticles indicating that not all cell types are transfected by these nanoparticles. Other prior work using chitosan-luciferase plasmid complexes (microspheres) also considered the mechanism of cell uptake to be endocytosis with the subsequent endosomal release of the plasmid and nuclear transport. Using one type of tumor cell, these processes were found to be affected by the molecular mass of chitosan, plasmid concentration, the stoichiometry of the complex, and serum concentration and pH of the transfection medium.

Confocal microscopy of the chondrocytes containing the FITC-labeled chitosan nanoparticles also revealed FITC-related fluorescence in the nucleus of the cells. A similar finding of FITC-related fluorescence in the nucleus of cells has been reported for FITC-labeled chitosan-plasmid complexes, on the order of 5-8 μm in diameter, used to transfect tumor cells. This indicated that the FITC released by the chitosan nanoparticles (in the present study) or from the chitosan microspheres in the cytoplasm had subsequently diffused into the nuclear compartment.

A notable finding of the present work was the expression of EGFP by the chondrocytes treated with nanoparticles incorporating the plasmids of both EGFP and OP-1. Control cultures containing the EGFP plasmid alone did not display fluorescence, indicating the likelihood that expression was the result of EGFP plasmid delivered by the nanoparticles. While the transfection efficiency was low, the very fact that some cells demonstrated the expression of EGFP suggested that the chitosan nanoparticles can gain entry into adult articular chondrocytes and the incorporated EGFP plasmid can gain entry into the nucleus and result in expression of the protein. EGFP was expressed very slowly, however, with chondrocytes beginning to synthesize the protein after 84 hours. Based on the other results showing that the nanoparticles gained entry into most of the cells after 24 hours, our supposition is that the complex between chitosan and the plasmid was so stable as to only allow a slow release of the plasmid in the cell. Based on this hypothesis, it would be of interest in future studies to prepare nanoparticles with a less positively charged polymer.

For chondrocytes transfected with nanoparticles incorporating pOP-1 alone, overexpression of OP-1 was not detected in the media by our ELISA assays; there may have been small amounts of OP-1 expressed that were below the detection limits of the ELISA assay. The fact that OP-1 expression was detected in cultures transfected with the GenePorter transfection reagent indicates that the OP-1 plasmid used was functional and able to transfect chondrocytes. One explanation for the findings may be that the interaction between OP-1 plasmid and the chitosan nanoparticles resulted in a slower release profile of the plasmid compared to the GenePorter transfection reagent. Overexpression may, therefore, only occur at later time points for nanoparticle-transfected cells. This supposition is supported by the observation that when the GenePorter reagent was used to transfect chondrocytes with the EGFP plasmid, there was earlier and stronger fluorescence compared to cells transfected with the EGFP-chitosan nanoparticles. That fluorescence was detected but no OP-1 was found in the medium in cultures transfected with nanoparticles incorporating the EGFP and OP-1 plasmids, respectively, may also indicate the importance of plasmid size on gene transfer to chondrocytes using chitosan nanoparticles. Future work will more exclusively investigate the efficacy of transfection using chitosan nanoparticles with varying plasmid sizes.

### Conclusions

In conclusion, chitosan nanoparticles incorporating plasmid OP-1 can be prepared with a range of diameters and morphologies by adjusting the chitosan:plasmid ratio. The OP-1 plasmid maintains its structural integrity after incorporation into the chitosan nanoparticle. A chitosan:plasmid weight ratio of 10:1 yields non-aggregating spherical nanoparticles of uniform size less than 250 nm. These particles can gain entry into adult articular chondrocytes and can result in expression of the plasmid carried by the nanoparticles, although expression also seems to be mediated by the size of incorporated plasmid.

The above also provides evidence that the particles are capable of gaining entry into renal cells and can result in expression of OP/BMP family proteins in renal cells.

1. A method of improving renal function in a mammal suffering from, or at risk of developing, at least partial renal failure or renal dysfunction, comprising administering to renal tissue of said mammal, a combination comprising a non-viral vector comprising a non-viral particulate carrier which carries a therapeutically effective amount of genetic material capable of expressing a renal function-enhancing Osteogenic Protein-1/Bone Morphogenic Protein-7 (OP-1/BMP-7) polypeptide in said renal tissue.

2. The method of claim 1 wherein said non-viral vector comprises polymer particles.

3. The method of claim 2 wherein said polymer is cationic.

4. The method of claim 3 wherein said polymer is natural.

5. The method of claim 4 wherein said polymer comprises chitosan.

6. The method of claim 2 wherein said particles have a size within a range of about 5-3,000 nm.

7. The method of claim 6 wherein said size is within a range of about 10-1,000 nm.

8. The method of claim 6 wherein said size is within a range of about 15-700 nm.

9. The method of claim 2 wherein said particles comprise nanoparticles.

10. The method of claim 1 wherein said genetic material comprises a DNA plasmid, and said polypeptide is OP-1/BMP-7.

11. The method of claim 1 wherein said combination is administered directly to a kidney of the mammal.

12. The method of claim 2 wherein said particles are in a composition wherein the particles are suspended in a pharmaceutically or veterinarily acceptable carrier.

13. The method of claim 12 wherein said composition is injected directly into a kidney of the mammal.

14. The method of claim 1 wherein said combination is present in a matrix which is contacted with said renal tissue.

15. The method of claim 14 wherein said matrix comprises collagen.

16. The method of claim 15 wherein said matrix comprises a collagen sponge.

17. The method of claim 15 wherein said collagen comprises collagen I, collagen II, collagen III or a combination thereof.

18. The method of claim 15 wherein said matrix is implanted in a kidney of said mammal, said matrix is attached to a surface of said kidney or a combination thereof.

19. The method of claim 9 wherein said matrix further comprises stem cells which are capable of differentiating into renal cells.

20. The method of claim 1 further comprising administering taurolidine, taurultam, a mixture thereof, or an equilibrium thereof, to said mammal.

21. A method of improving renal function in a mammal suffering from, or at risk of developing, at least partial renal failure or renal dysfunction, comprising administering to renal tissue of said mammal a combination comprising a non-viral vector comprising chitosan particles having a size within a range of about 15-700 nm, the particles incorporating a therapeutically effective amount of plasmid DNA capable of expressing a renal function-enhancing OP-1/BMP-7 polypeptide in said renal tissue.

22. The method of claim 21 wherein the plasmid DNA is encapsulated in the chitosan particles.

23. The method of claim 2 wherein said genetic material is encapsulated in said particles.
